(19) United States
(12) Patent Application Publication
(10) Pub. No.: US 2003/0225272 A1
(43) Pub. Date: Dec. 4, 2003

(54) Novel mmp-2/mmp-9 inhibitors

(76) Inventors: Anne Romanic Arnold, Wynnewood, PA (US); Balan Chenera, Thousand Oaks, CA (US); Gerald R. Girard, Bensalem, PA (US); Joseph Weinstock, Wayne, PA (US)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/276,940

(22) PCT Filed: May 24, 2001
(86) PCT No.: PCT/US01/16867

(51) Int. Cl. C07D 265/28; C07C 237/20

(52) U.S. Cl. 544/162; 554/51

(57) ABSTRACT

Novel MMP-2/MMP-9 inhibitors and methods of using them are provided.

### FIELD OF THE INVENTION

The present invention relates to novel, dual inhibitor of matrix metalloproteinase-2 (herein “MMP-2”) and matrix metalloproteinase-9 (herein “MMP-9”). The present invention further relates to methods for treating pain in a patient, comprising administering to the patient a pain-reducing effective amount of a present compound.

### BACKGROUND OF THE INVENTION

The extracellular matrix (ECM) is a multifunctional complex of proteins and proteoglycans assembled in a highly organized manner that contributes to the structural integrity of cells and tissue within an organ system. The basement membrane, which provides structural support to the vasculature, is comprised of ECM molecules such as type IV collagen, laminin, and fibronectin. Various factors are involved in maintaining the integrity of the ECM and the tissues it supports. However, in certain pathological circumstances, the ECM is modulated such that the structure of the tissue becomes damaged or destroyed. The matrix metalloproteinases (MMPs) are a group of zinc-dependent enzymes that degrade the molecules of the extracellular matrix. Two members of the MMP family, MMP-2 (72 kDa gelatinase/Gelatinase A) and MMP-9 (92 kDa gelatinase/Gelatinase B), degrade the ECM components of the basement membrane. Their substrates include types IV and V collagen, fibronectin, elastin, and denatured interstitial collagen s. Matrix degradation attributed to these proteinases has been shown to play an important role in the progression of diseases such as atherosclerosis, inflammation, stroke, and tumor growth and metastasis.

Nerve injury caused by constriction results in ischemia of the nerve tissue and, ultimately, neuronal cell death. Nerve injury following constriction is primarily a result of the decrease in blood flow and of energy depletion due to compression of microvessels which supply the nervous tissue. These events cause the nerve tissue to become infarcted, with contributions from excitotoxicity, enzyme activation, edema, and inflammation. A significant inflammatory response occurs following nerve injury. For example, neutrophils infiltrate the damaged tissue and contribute to the nerve injury, further exacerbating the injury response. Further, researchers have demonstrated that neutrophils utilize MMPs for their migration. It is believed that MMP inhibition would prevent or ameliorate the tissue damage that occurs following nerve injury. Further MMP inhibition would prevent or reduce the degree of inflammatory cell infiltration into the damaged tissue.

Clearly, there is a need for identification and characterization of dual antagonists of MMP-2 and MMP-9 that play a role in preventing, ameliorating or correcting stroke; hemorrhage; reperfusion injury; cerebral ischernia; cerebral infarction; enhanced or exaggerated sensitivity to pain, such as hyperalgesia, causalgia and allodynia; acute pain; burn pain; atypical facial pain; neuropathic pain; back pain; complex regional pain syndromes I and II; arthritic pain; sports injury pain; pain related to viral infection, e.g., HIV, post-polio syndrome, and post-herpetic neuralgia; phantom limb pain; labor pain; cancer pain; post-chemotherapy pain; post-stroke pain; post-operative pain; physiological pain; inflammatory pain; acute inflammatory conditions/visceral pain, e.g., angina, irritable bowel syndrome (IBS), and inflammatory bowel disease; neuropathic pain; neuralgia; painful diabetic neuropathy; traumatic nerve injury; spinal cord injury; and tolerance to narcotics or withdrawal from narcotics, among others.

In one aspect, the present invention relates novel MMP-2/MMP-9 inhibitors and to a method for treating pain in a patient, said method comprising the step of administering to the patient a pain-treating effective amount of a present compound in combination with a carrier, wherein the patient is suffering from enhanced or exaggerated sensitivity to pain, such as hyperalgesia, causalgia and allodynia; acute pain; burn pain; atypical facial pain; neuropathic pain; back pain; complex regional pain syndromes I and II; arthritic pain; sports injury pain; pain related to viral infection, e.g., HIV, post-polio syndrome, and post-herpetic neuralgia; phantom limb pain; labor pain; cancer pain; post-chemotherapy pain; post-stroke pain; post-operative pain; physiological pain; inflammatory pain; acute inflammatory conditions/visceral pain, e.g., angina, irritable bowel syndrome (IBS), and inflammatory bowel disease; neuropathic pain; neuralgia; painful diabetic neuropathy; traumatic nerve injury; spinal cord injury; and tolerance to narcotics or withdrawal from narcotics.

In a second aspect, the invention relates to the present compounds and a method for treating nerve tissue damage in a patient in need thereof, said method comprising the step of administering an effective nerve tissue damage-reducing amount of present compound in combination with a carrier, wherein the patient is suffering from stroke; hemorrhage; reperfusion injury; cerebral ischemia; cerebral infarction; enhanced or exaggerated sensitivity to pain, such as hyperalgesia, causalgia and allodynia; acute pain; burn pain; atypical facial pain; neuropathic pain; back pain; complex regional pain syndromes I and II; arthritic pain; sports injury pain; pain related to viral infection, e.g., HIV, post-polio syndrome, and post-herpetic neuralgia; phantom limb pain; labor pain; cancer pain; post-chemotherapy pain; post-stroke pain; post-operative pain; physiological pain; inflammatory pain; acute inflammatory conditions/visceral pain, e.g., angina, irritable bowel syndrome (IBS), and inflammatory bowel disease; neuropathic pain; neuralgia, painful diabetic neuropathy; traumatic nerve injury; spinal cord injury; and tolerance to narcotics or withdrawal from narcotics.

In a third aspect, the invention relates to the present compounds and a method for treating a patient suffering from a disease selected from the group consisting of: stroke, hemorrhage, reperfusion injury, cerebral ischemia,, and cerebral infarction, said method comprising the step of administering an effective amount of a present compound

### SUMMARY OF THE INVENTION

The present invention involves novel compounds represented by Formula (I), hereinbelow and its use as an MMP2/9 inhibitor.

The present invention further provides methods for inhibiting MMP2/9 in an animal, including humans, which comprises administering to a subject in need of treatment an effective amount of a compound of Formula (I), as indicated hereinbelow.

### DETAILED DESCRIPTION OF THE INVENTION

The compounds useful in the present methods are selected from Formula (I), hereinbelow.

Compounds of formula (I) have the following structure:

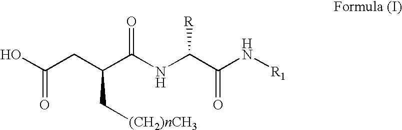

wherein:

R is selected from a group consisting of alkyl, aryl, arylalkyl, heteroaryl, heteroalkylaryl,alkylthioalkyl, hydroxyalkyl, and aminoalkyl; and

R₁ is selected from the group consisting of alkyl, cycloalkyl, aryl, heteroaryl, arylalkyl, heteroarylalkyl, aminoalkyl, and (N-substitutedaminosulfonyl) amino alkylamino, wherein the amino of the aminoalkyl may be unsubstituted, mono or disubstituted with an alkyl or aryl group or be part of a heterocyclic ring, and the N-substitutedamino of the (N-substitutedaminosulfonyl) may also be be unsubstituted, mono or disubstituted with an alkyl or aryl group or be part of a heterocyclic ring.

The aryl groups of R and R₁ may be substituted with groups such as alkyl, alkenyl, arylalkyl, acyl, aroyl, haloalkyl, halo, carboxy, carboalkoxy, carbamyl, alkylcarbamyl, arylcarbamyl, cyano, alkoxy, hydroxyl, phenylazo, anino, nitro, alkylamino, arylamino, arylalkylamino, acylamino, aroylamino, alkylthio, arylalkylthio, arylthio, alkysulfinyl, arylsulfinyl, arylalkylsulfinyl, alkylsulfonyl, arylsulfonyl, arylalkylsulfonyl, sulfamyl, arylsulfonamido, or alkylsulfonamido.

As used herein, “alkyl” refers to an optionally substituted hydrocarbon group joined together by single carbon-carbon bonds. The alkyl hydrocarbon group may be linear, branched or cyclic, saturated or unsaturated. Preferably, the group is unsubstituted. Preferably, the group is saturated. Preferred alkyl moieties are C1-5 alkyl.

As used herein, “aryl” refers to an optionally substituted aromatic group with at least one ring having a conjugated pi-electron system, containing up to two conjugated or fused ring systems. “Aryl” includes carbocyclic aryl, heterocyclic aryl and biaryl groups, all of which may be optionally substituted. Preferred aryl moieties are phenyl, unsubstituted, monosubstituted, disubstituted or trisubstituted.

Preferred compounds, having formula (I), useful in the present invention are selected from the group consisting of:

N-[2(R)-Nonylsuccinic acid]-L-tyrosine-N-3-(N-morpholino)propylamide

N-[2(R)-Nonylsuccinic acid]-L-phenylglycine-N-3-(N-morpholino)propylamide

N-[2(R)-Nonylsuccinic acid]-L-leucine-N-3-(N-morpholino)propylamide

N-[2(R)-Nonylsuccinic acid]-L-methionine-N-3-(N-morpholino)propylamide

N-[2(R)-Nonylsuccinic acid]-L-tyrosine-N-2-(N-morpholino)ethylamide

N-[2(R)-Nonylsuccinic acid]-L-phenylalanine-N-3-(N-morpholino)propylamide

N-[2(R)-Nonylsuccinic acid]-L-valine-N-2-(N-morpholino)ethylamide

N-[2(R)-Nonylsuccinic acid]-L-tyrosine-N-(4-methoxyphenyl)amide

N-[2(R)-Nonylsuccinic acid]-L-phenylalanine-N-(4-methoxyphenyl)amide

N-[2(R)-Nonylsuccinic acid]-L-norvaline-N-(4-methoxyphenyl)amide

N-[2(R)-Nonylsuccinic acid]-L-arginine-N-(4-methoxyphenyl)amide

N-[2(R)-Nonylsuccinic acid]-L-phenylglycine-N-methylamide

N-[2(R)-Nonylsuccinic acid]-L-tyrosine-N-cyclopentylamide; and

N-[2(R)-Nonylsuccinic acid]-L-tyrosine-N-3-dimethylaminopropylamide

Also included in the present invention are pharmaceutically acceptable salts and complexes. Preferred are the zinc, copper, nickel, cobalt and rhodium complexes, hydrochloride, hydrobromide and trifluoroacetate salts. The compounds of the present invention may contain one or more asymmetric carbon atoms and may exist in racemic and optically active forms. All of these compounds and diastereomers are contemplated to be within the scope of the present invention.

Synthesis

A synthesis of N-[2(R)-nonylsuccinic acid]-L-phenylglycine-N-methylamide was carried out as shown in Scheme 1 by reacting 4(S)Benzyl-2-oxazolidine with butyl lithium to give the nitrogen anion and then reaction of this with undecanoyl chloride to give (S)-4-benzyl-3-undecanoyl-oxazolidin-2-one (2). This was converted to the anion by reaction with lithium diisopropylamide and quenched with t-butyl bromoacetate to give give 4(S)-benzyl-3-[2-(R)-[(tert-butoxycarbonyl)methyl]undecanoyl]-2-oxazolidinone (3) which was purified by chromatography. This product was converted to 2-(R)-[(tert-butoxycarbonyl)methyl]undecanoic acid (4) by hydrolysis with lithium hydroxide in the presence of hydrogen peroxide. L-Phenylglycine-N-methylarnide was prepared by reaction of the methyl ester of L-phenylglycine with methyl amine, and this condensed with 4 in a standard amide forming reaction to give 2-(R)-[(tert-butoxycarbonyl)methyl]undecanoyl-L-phenylglycine-N-methylamide (5). After purification by chromatography this was hydrolyzed by treatment with 90% trifluoroacetic acid to give the desired N-[2(R)-nonylsuccinic acid]-L-phenylglycine-N-methylamide which was crystallized from acetonitrile.

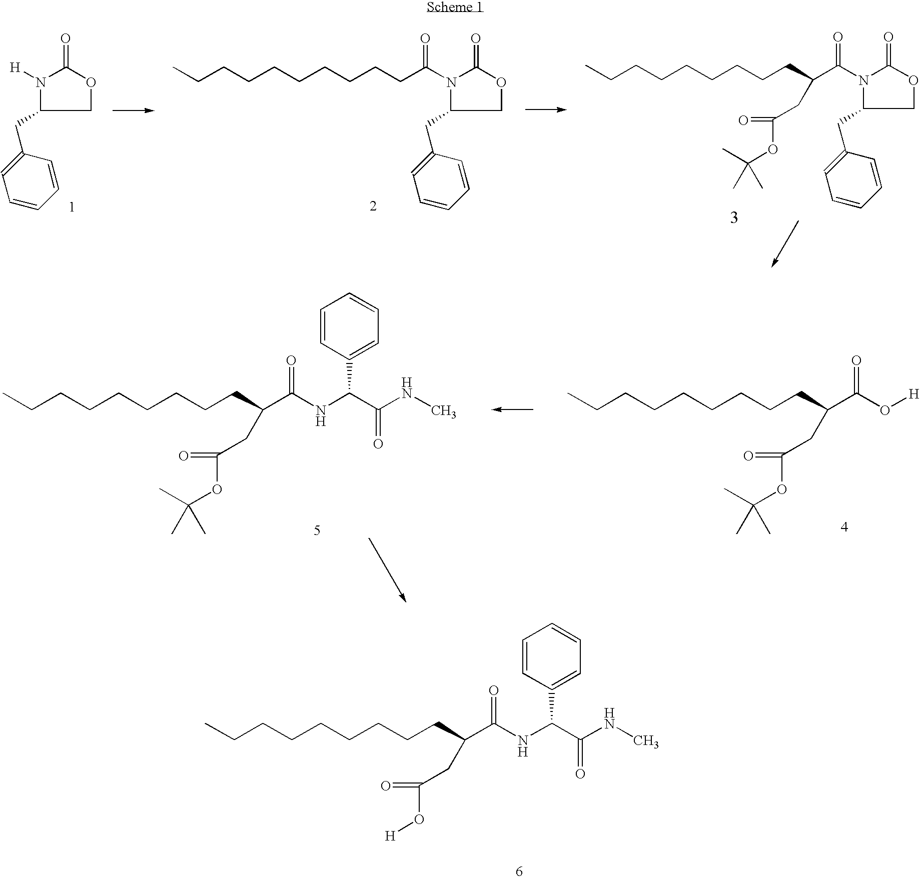

The compounds of this invention may also be prepared in an array format on polystyrene resin. To prepare N-[2(R)-Nonylsuccinic acid]-L-phenylalanine-N-3-(N-morpholino)propylamide N-(3-aminopropyl)morpholine was condensed with (4-Formyl-3,5-dimethoxyphenoxy)methyl polystyrene resin using sodium triacetoxyborohydride as the reducing agent. The product was coupled with (S)-Fmoc-phenylalanine using 1-hydroxy-7-azabenzotriazole (0.25 mmol) and di-isopropylcarbodiinide. The Fmoc proteacting group was removed with piperidine and the resulting product coupled with R-2-nonylsuccinic acid, 4-t-butyl ester using 1-hydroxy-7-azabenzotriazole and di-isopropylcarbodiimide. N-[2(R)-Nonylsuccinic acid]-L-phenylalanine-N-3-(N-morpholino)propylamide was obtained by treating the resin with trifluoroacetic acid and purification by automated preparative HPLC. LCMS analysis found that the product had the anticipated molecular weight of 517.

By a similar procedure compounds were prepared using as amines methyl amine, 2-aminomethylpyridine, dimethylaminopropyl amine, 4-methoxyphenethyl amine, cyclopentyl amine, p-anisidine, 4-(3-aminopropyl)morpholine, and 2-aminoethylmorpholine and using as FMOC aminoacids Fmoc-glycine, Fmoc-serine, Fmoc-valine, Fmoc-norvaline, Fmoc-leucine, Fmoc-isoleucine, Fmoc-phenylalanine, t-BuO-Fmoc-tyrosine, Fmoc-methionine, Fmoc-D-homophenylalanine, Fmoc-phenyglycine, and Fmoc-lysine. Similar compounds may be prepared by using other amines for the reductive amination and other FMOC aminoacids.

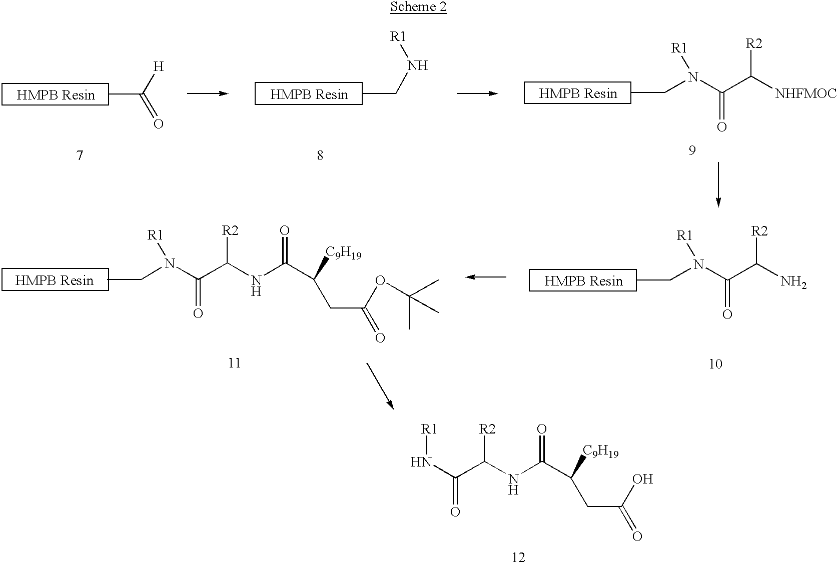

With appropriate manipulation and protection of any chemical functionality, synthesis of the remaining compounds of Formula (I) and (II) is accomplished by methods analogous to those above and to those described in the Experimental section.

In order to use a compound of the Formula (I) or a pharmaceutically acceptable salt thereof for the treatment of humans and other mammals it is normally formulated in accordance with standard pharmaceutical practice as a pharmaceutical composition.

As used herein, “treatment” of a disease includes, but is not limited to prevention, retardation and prophylaxis of the disease. The present compounds are useful for the treatment of diseases including but not limited to: stroke; hemorrhage; reperfusion injury; cerebral ischemia; cerebral infarction; enhanced or exaggerated sensitivity to pain, such as hyperalgesia, causalgia and allodynia; acute pain; burn pain; atypical facial pain; neuropathic pain; back pain; complex regional pain syndromes I and II; arthritic pain; sports injury pain; pain related to viral infection, e.g., HIV, post-polio syndrome, and post-herpetic neuralgia; phantom limb pain; labor pain; cancer pain; post-chemotherapy pain; post-stroke pain; post-operative pain; physiological pain; inflammatory pain; acute inflammatory conditions/visceral pain, e.g., angina, irritable bowel syndrome (IBS), and inflammatory bowel disease; neuropathic pain; neuralgia; painful diabetic neuropathy; traumatic nerve injury; spinal cord injury; and tolerance to narcotics or withdrawal from narcotics.

Compounds of Formula (I) or (II) and their pharmaceutically acceptable salts may be administered in a standard manner for the treatment of the indicated diseases, for example orally, parenterally, sub-lingually, dermally, transdermally, rectally, via inhalation or via buccal administration.

Compositions of Formula (I) or (II) and their pharmaceutically acceptable salts which are active when given orally can be formulated as syrups, tablets, capsules, creams and lozenges. A syrup formulation will generally consist of a suspension or solution of the compound or salt in a liquid carrier for example, ethanol, peanut oil, olive oil, glycerine or water with a flavoring or coloring agent. Where the composition is in the form of a tablet, any pharmaceutical carrier routinely used for preparing solid formulations may be used. Examples of such carriers include magnesium stearate, terra alba, talc, gelatin, acacia, stearic acid, starch, lactose and sucrose. Where the composition is in the form of a capsule, any routine encapsulation is suitable, for example using the aforementioned carriers in a hard gelatin capsule shell. Where the composition is in the form of a soft gelatin shell capsule any pharmaceutical carrier routinely used for preparing dispersions or suspensions may be considered, for example aqueous gums, celluloses, silicates or oils, and are incorporated in a soft gelatin capsule shell.

Typical parenteral compositions consist of a solution or suspension of a compound or salt in a sterile aqueous or non-aqueous carrier optionally containing a parenterally acceptable oil, for example polyethylene glycol, polyvinylpyrrolidone, lecithin, arachis oil or sesame oil.

Typical compositions for inhalation are in the form of a solution, suspension or emulsion that may be administered as a dry powder or in the form of an aerosol using a conventional propellant such as dichlorodifluoromethane or trichlorofluoromethane.

A typical suppository formulation comprises a compound of Formula (I) or (II) or a pharmaceutically acceptable salt thereof which is active when administered in this way, with a binding and/or lubricating agent, for example polymeric glycols, gelatins, cocoa-butter or other low melting vegetable waxes or fats or their synthetic analogs.

Typical dermal and transdermal formulations comprise a conventional aqueous or non-aqueous vehicle, for example a cream, ointment, lotion or paste or are in the form of a medicated plaster, patch or membrane.

Preferably the composition is in unit dosage form, for example a tablet, capsule or metered aerosol dose, so that the patient may administer a single dose.

Each dosage unit for oral administration contains suitably from 0.1 mg to 500 mg/Kg, and preferably from 1 mg to 100 mg/Kg, and each dosage unit for parenteral administration contains suitably from 0.1 mg to 100 mg/Kg, of a compound of Formula(I) or (II) or a pharmaceutically acceptable salt thereof calculated as the free acid. Each dosage unit for intranasal administration contains suitably 1-400 mg and preferably 10 to 200 mg per person. A topical formulation contains suitably 0.01 to 5.0% of a compound of Formula (I) or (II).

The daily dosage regimen for oral administration is suitably about 0.01 mg/Kg to 40 mg/Kg, of a compound of Formula (I) or (II) or a pharmaceutically acceptable salt thereof calculated as the free acid. The daily dosage regimen for parenteral administration is suitably about 0.001 mg/Kg to 40 mg/Kg, of a compound of Formula (I) or (II) or a pharmaceutically acceptable salt thereof calculated as the free acid. The daily dosage regimen for intranasal administration and oral inhalation is suitably about 10 to about 500 mg/person. The active ingredient may be administered from 1 to 6 times a day, sufficient to exhibit the desired activity.

No unacceptable toxicological effects are expected when compounds of the present invention are administered in accordance with the present invention.

The biological activity of the compounds of Formula (I) or (II) are demonstrated by the following tests:

### EXAMPLE 3

MMP-2/MMP-9 Screening Assay Protocol

A high-throughput, 96-well screen was used to measure MMP-9 activity and to detect potential inhibitors of MMP-9. The screen is a quenched fluorescence assay. The components of the assay include purified recombinant human MMP-9 (generated by. SB, 3 nM final concentration) and a fluorogenic peptide substrate (Peptides International, Louisville, Ky., 10 (M final concentration) incubated in the presence or absence of compound. Briefly, enzyme activity is measured after 30 minutes incubation at 37(C. and quantitated using a peptide substrate, (Dnp-Pro-Cha-Gly-Cys(Me)-His-Ala-Lys(NMa)-NH2 or (2,4-Dinitrophenyl-L-Prolyl-L-Cyclohexylalanyl-Glycyl(-Methyl-L-Cysteinyl-L-Histidyl-L-Alanyl-N(-Methylenthranoyl-L-Lysine Amide) containing a fluorophore, Nma, on one end of the peptide and a quencher, Dnp, on the other end. When the peptide is intact, the fluorophore is quenched. When the peptide is cleaved by MMP-9, the quencher is dissociated from the fluorophore and a fluorescent signal is emitted that can easily be detected using a fluorescent plate reader. The universal cleavage site within the peptide that is recognized by MMP-1, -2, -3, -9 and -13 is the Gly-Cys bond. Compounds that exhibit an IC50 for MMP-9 that is less than 1 (m were subjected to additional screens using purified recombinant human MMP-2 (generated by SB, 10 nM), MMP-13 (Chemicon, Temecula, Calif.), MMP-3 (Biogenesis, Sandown, N.H.) and MMP-1 (Biogenesis, Sandown, N.H.) to confirm selectivity for MMP-9. These screens were conducted using the same fluorogenic peptide substrate as that described above for MMP-9. Using this screening protocol, N-[2(R)-(n-Nonyl)succinyl]-L-phenylglycine-N-methylamide, was identified as a dual MMP-2/MMP-9 inhibitor. The synthesis of this compound is described in detail in Example 3.

Determination of Ic50 for Carboxylic Acid Compounds Inhibition of MMP-9 & MMP-2.

Background

A 96-well quenched fluorescence assay was used to measure MMP-9 and MMP-2 activity and to detect potential inhibitors of MMP-9 and MMP-2. The components of the assay included purified recombinant human MMP-2 or -9 and a fluorogenic peptide substrate incubated in the presence or absence of compound. Compounds were initially screened at 1 uM against MMP-9 and those compounds that inhibited MMP-9>95% were subjected to additional screens against purified recombinant human MMP-2, MMP-1 and MMP-3. For these additional screens, an IC50 value was determined.

MMP-9 and MMP-2 Screen

Enzyme activity was measured and quantitated using a peptide substrate, 2,4-Dinitrophenyl-L-Prolyl-L-Cyclohexylalanyl-Glycyl(-Methyl-L-Cysteinyl-L-Histidyl-L-Alanyl-N(-Methylenthranoyl-L-Lysine Amide, (Dnp-(Pro-Cha-Gly-Cys(Me)-His-Ala-Lys(NMa)-NH2, Peptides International cat #SDP-3815), containing a fluorophore, Nma, on one end of the peptide and a quencher, Dnp, on the other end (Bickett et al., 1993). When the peptide is intact, the fluorophore is quenched. When the peptide is cleaved by an MMP, the quencher is dissociated from the fluorophore and a fluorescent signal can be detected using a fluorescent plate reader (Ex. 355 nm Em. 460 nM). The cleavage site within the peptide that is recognized by MMP-2 and MMP-9 is the Gly-Cys bond.

A 3 uM (500 ul )working stock solution was made for each compound. All working solutions were made in an assay buffer consisting of 100 mM Tris; pH 7.5, 100 mM NaCl; 10 mM CaCl2; 0.01% NaN3. From this working stock, logarithmic dilutions were made with the assay buffer and each compound was tested in triplicate at 1 uM, 300 nM, 100 nM, 30 nM, 10 nM, 3 nM and 1 nM. The concentration of the peptide substrate was 10 uM. The concentration of MMP-9 used in the assay was 0.3 nM and for MMP-2 the concentration used was 10 nM. At these concentrations, peptide cleavage was maximal and reached a plateau phase at 30 minutes of incubation at 37(C. Fluorescence was measured at t=0 and t=30 minutes at 37(C. The delta fluorescence for each reaction, relative to the vehicle control, was determined and percent inhibition was calculated. These values were then plotted, relative to compound concentration, and the IC50 was extrapolated.

MMP-1 Screen

MMP-1 was obtained as the active form from T. Cawston, London, UK. To assay for MMP-1 activity and compound inhibition, 10 ul of 20% DMSO in assay buffer or 10 ul of compound inhibitor at 10× final concentration in 20% DMSO was added to a 96-well plate. Then, 70 ul of assay buffer, 10 ul of SDP-3815 peptide substrate (Peptides International) at a concentration of 500 uM in 10% DMSO in assay buffer, and 10 ul of MMP-1 (a final concentration of 24 ug/ml in assay buffer) was added to each well.

Fluorescence was measured at 10-min intervals for up 30 min with a fluorescent plate reader (360/460 mm filter pair). if non-linear at end of time, use linear portion to determine slope. To determine percent MMP-1 inhibition, the slope of fluorescence signal generated vs. time was plotted.

MMP-3 Screen

Pro-MMP-3 (prostromelysin) was purchased from Biogenesis (cat#5980-0357) 230 ug/ml and activated according to Lark et al, Connective Tissue Res. 25, 52 (1990). Briefly, to 5 ul of 230 ug/ml pro-stromelysin, 5 ul of 160 nM trypsin (Fluka) in 0.15 M Tris Cl, 15 mM CaCl2, 0.2 M NaCl, pH 7.6 (assay buffer) was added. The reaction mixture was allowed to incubate for 30 min at 37° C., after which 3.3 ul of ⅙ dilution (in 0.5 M NaCl) of soybean trypsin inhibitor on agarose beads (Sigma), equivalent to 100-fold excess over trypsin, was added. This reaction mixture was then incubated for another 30 min at 37° C. and then centrifuged for 5 min at 14,000 rpm (microfuge) to spin down the beads. The sample was then stored on ice for immediate use or aliquoted and stored at −80° C. The final concentration of MMP-3 was 1.5 uM.

To assay for MMP-3 activity and compound inhibition, 10 ul of 20% DMSO in assay buffer or 10 ul of compound inhibitor at 10× final concentration in 20% DMSO was added to a 96-well plate. Then, 70 ul of assay buffer, 10 ul of NFF-3 peptide substrate (Peptides International) at a concentration of 500 uM in 10% DMSO/assay buffer, and 10 ul of stromelysin (a final concentration of 75 nM in assay buffer) was added to each well.

Fluorescence was measured at 10-min intervals for up to 1 hr using a fluorescent plate reader (320/405 nm filter pair). To determine percent MMP-3 inhibition, the slope of fluorescence signal generated vs. time was plotted.

The following examples are illustrative but not limiting of the embodiments of the present invention.

### EXAMPLE 1

N-[2(R)-Nonylsuccinic acid]-L-phenylglycine-N-methylamide

(S)-4-Benzyl-3-undecanoyloxazolidine-2-one (2). A solution of 21.5 g (0.122 ml) of 4(S)-benzyloxazolidine-2-one (1) in THF(250 ml) was cooled to −78(C. and treated with 61 ml (0.128 mol) of 2.1M n-butyl lithium in hexane. The mixture was stirred for 45 min at −78(C. and then a solution of 27.5 g (0.134 mole) of undecanoyl chloride in 50 ml of THF added dropwise. The mixture was stirred at −78(C. for 1 hour and then allowed to warm to ambient temperature over 18 hours. Twenty ml of 0.1N HCl was added dropwise and then ethyl acetate added to allow phase separation. The organic layer was washed with H2O, saturated NaHCO3 and saturated saline. Concentration under vacuum gave the product. LC/MS: Calc. MW 345; Found: M+1 346; RT 3.20 min. 1×40 mm C18 column, 4.5% to 90% CH3CN (0.02% TFA) in 3.2 min with 0.4 kmin hold and 1.4 min re-equilibration, UV and MS detection.

R-3-[1-((1S)-4-Benzyl-2-oxo-oxazolidin-3-yl)-methanoyl]-dadecanoic acid, tert-butyl ester (3). A solution of 18.1 ml (13.07 g, 0.129 mol) of diisopropylamine in 200 ml of THF was treated with 48.2 ml (0.121 mol) of 2.5M n-butyl lithium in THF. After 30 minutes a solution of 40.69 g (0.117 mol) of S-4-benzyl-3-undecanoyl-oxazolidin-2-one in 150 ml of THF was added and the mixture stirred at −78(C. for 90 minutes. Then 20.75 ml (27.4 g, 0.14 mol) of t-butyl bromoacetate was added followed by 50 ml of THF from the same dropping funnel. The mixture was allowed to stand at ambient temperature for 18 hours and then 300 ml. of 0.5N HCl added and the mixture extracted with ethyl acetate. The organic layer was washed with H2O, 5% NaHCO3, saturated saline, and then dried over MgS04. Concentration under vacuum gave the crude product which was chromatographed over Si02 using 5% ethyl acetate/hexane to elute the product. LC/MS: Calc. MW 459; Found: M+1 460, RT 3.45 min.

R-2-Nonyl-succinic acid, 4-t-butyl ester (4). A solution of 7.9 g (17.2 mmol) of 3 in 175 ml of THF was cooled to 0(C. and 9 ml (80 mmol) of 30% hydrogen peroxide and 1.24 g (29.5 mmol) of lithium hydroxide monohydrate dissolved in 50 ml of H2O added dropwise. The mixture was then stirred at ambient temperature for two hours, cooled to 0(C. and 5.2 g (75 mmol) of sodium nitrite added. After 30 minutes the mixture was concentrated under vacuum and more water added. The basic solution was washed with ether, the pH adjusted with 12N HCl to 2.5, and extracted with EtOAc three times. The organic solution was dried over MgS04 and concentrated under vacuum to give the product which had the expected NMR spectrum. LC/MS: Calc. MW 300; Found: M+1 301, RT 2.90 min.

N-[t-Butyl-2(R)-nonylsuccinic acid]-L-phenylglycine-N-methylamide (5). A solution of L-phenylglycine-N-methylamide (7.25 g, 44.2 mmol) and 4 (13.27 g, 44.2 mmol) in 125 of DMF was treated with EDC-HCl (8.47 g, 44.2 mmol) and stirred for 48 hours. The reaction mixture was diluted with EtOAc and washed with 0.5N HCl, H20, saturated NaHCO3, and saturated saline. It was dried over MgSO4 and concentrated. The resulting product was purified by chromatography (SiO2, elution with 30% ETOAc/hexane). LC/MS: Calc. MW 446; Found: M+1 447, RT 3.02 min.

N-[2(R)-Nonylsuccinic acid]-L-phenylglycine-N-methylamide (6). A solution of 5 (10.36 g, 23.2 mmol) in 80 ml of 90% TFA was stirred for 2.5 hours and then concentrated under vacuum. The residue was triturated with EtOAc and then concentrated under vacuum. Addition of CH3CN gave crystals which were collected and washed with fresh CH3CN. A second crop was obtained by concentration of the combined mother liquor and washings under vacuum and dissolving the residue in EtOAc which was washed with H2O, dried over MgSO4, and concentrated under vacuum. Addition of CH3CN to the residue gave additional crystals which were combined with the first crop to give colorless crystals, mp156-158(C. LC/MS: Calc. MW 390; Found: M+1 391, RT 2.17 min. Anal. Calcd: C, 67.66; H, 8.78; N, 7.17. Found: C, 67.87; H, 9.16; N, 7.15.

### EXAMPLE 2

N-[2(R)-Nonylsuccinic acid]-L-phenylalanine-N-3-(N-morpholino)propylamide Prepared on Polystyrene Resin.

Reductive Amination of Resin.

(4-Formyl-3,5-dimethoxyphenoxy)methyl polystyrene resin (Polymer Laboratories, 1.82 mmol/gm., 10 g) was suspended in a mixture of N-methylpyrrolidinone(100 mL) and acetic acid(25 mL) in a large shaker vessel. N-3-aminopropylmorpholine (0.1 mol) was added and the mixture shaken at room temperature for 1 h. Sodium triacetoxyborohydride(0.05 mol) was then added in N-methylpyrrolidine(50 mL) and the mixture was allowed to shake at room temperature overnight. The resin was then filtered, washed with 1:1 DMF/water(3×), DMF(3×), and dichloromethane(4×).

Coupling of Fmoc-amino acid

The reductively aminated resin (50 mg) was suspended in 1 mL of N-methylpyrrolidinone. To this was added (S)-Fmoc-phenylalanine (0.25 mmol), 1-hydroxy-7-azabenzotriazole (0.25 mmol) and di-isopropylcarbodiimide (0.25 mmol). The reaction mixture was shaken at room temperature overnight, filtered, washed with DMF(4×) and the coupling repeated once more. The resin was filtered, washed with DMP(4×) and dichloromethane(4×).

Removal of FMOC Group.

The product from the above step was treated with 20% piperidine in DMF (1.5 mL) and agitated for one hour. The resin was washed with DMF(4×).

Coupling of R-2-nonylsuccinic acid, 4-t-butyl ester.

The product from the above step was suspended in N-methylpyrrolidinone(1 mL). To this was added R-2-nonylsuccinic acid, 4-t-butyl ester (0.25 mmol), 1-hydroxy-7-azabenzotriazole (0.25 mmol) and di-isopropylcarbodiimide (0.25 mmol). The reaction mixture was shaken at room temperature for 16 h, filtered, and the resin washed with DMF(4×), Methanol(4×) and dicloromethane(4×).

TFA cleavage to yield N-[2(R)-Nonylsuccinic acid]-L-phenylalanine-N-3-(N-morpholino)propylamide (12).

The resin obtained from the previous step was treated with trifluoroacetic acid(1.5 mL) and agitated for 8 h, filtered, and washed with dichloromethane. The combined filtrate was concentrated and subjected to purification using automated preparative HPLC and concentrated in a vacuum centrifuge The residue when analyzed by LCMS gave a molecular weight of 517.

Formulations for pharmaceutical use incorporating compounds of the present invention can be prepared in various forms and with numerous excipients. Examples of such formulations are given below.

### EXAMPLE 3

Inhalant Formulation

A compound of Formula I or II, (1 mg to 100 mg) is aerosolized from a metered dose inhaler to deliver the desired amount of drug per use.

### EXAMPLE 4

Tablet Formulation

| Tablets/Ingredients | | Per Tablet | |
|---|---|---|---|
| 1. | Active ingredient | 40 | mg |
| | (Cpd of Form. I or II) | | |
| 2. | Corn Starch | 20 | mg |
| 3. | Alginic acid | 20 | mg |
| 4. | Sodium Alginate | 20 | mg |
| 5. | Mg stearate | 1.3 | mg |

Procedure For Tablet Formulation:

Ingredients 1, 2, 3 and 4 are blended in a suitable mixer/blender. Sufficient water is added portion-wise to the blend with careful mixing after each addition until the mass is of a consistency to permit its conversion to wet granules. The wet mass is converted to granules by passing it through an oscillating granulator using a No. 8 mesh (2.38 mm) screen. The wet granules are then dried in an oven at 140° F. (60° C.) until dry. The dry granules are lubricated with ingredient No. 5, and the lubricated granules are compressed on a suitable tablet press.

### EXAMPLE 8

Parenteral Formulation

A pharmaceutical composition for parenteral administration is prepared by dissolving an appropriate amount of a compound of Formula I or II in polyethylene glycol with heating. This solution is then diluted with water for injections Ph Eur. (to 100 ml). The solution is then rendered sterile by filtration through a 0.22 micron membrane filter and sealed in sterile containers.

All publications, including but not limited to patents and patent applications cited in this specification are herein incorporated by reference as if each individual publication were specifically and individually indicated to be incorporated by reference as though fully set forth.

What is claimed is:

1. A compound according to formula (I) 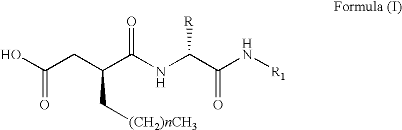
wherein:
R is selected from a group consisting of alkyl, aryl, arylalkyl, heteroaryl, heteroalkylaryl,alkylthioalkyl, hydroxyalkyl, and aminoalkyl; and
R₁ is selected from the group consisting of alkyl, cycloalkyl, aryl, heteroaryl, arylalkyl, heteroarylalkyl, aminoalkyl, and (N-substitutedaminosulfonyl) aminoalkylamino, wherein the amino of the aminoalkyl may be unsubstituted, mono or disubstituted with an alkyl or aryl group or be part of a heterocyclic ring, and the N-substitutedamino of the (N-substitutedaminosulfonyl) may also be unsubstituted, mono or disubstituted with an alkyl or aryl group or be part of a heterocyclic ring;
and n is between 8 and 16, and the chain consisting of (CH₂)nCH₃ may be interrupted by sulfur and/or oxygen atoms with the sulfur carrying 0-2 oxygen atoms.

2. A compound according to claim 1 selected from the group consisting of:
N-[2(R)-Nonylsuccinic acid]-L-tyrosine-N-2-(N-morpholino)ethylamide;
N-[2(R)-Nonylsuccinic acid]-L-phenylalanine-N-3-(N-morpholino)propylamide;
N-[2(R)-Nonylsuccinic acid]-L-valine-N-2-(N-morpholino)ethylamide;
N-[2(R)-Nonylsuccinic acid]-L-tyrosine-N-(4-methoxyphenyl)amide;
N-[2(R)-Nonylsuccinic acid]-L-phenylalanine-N-(4-methoxyphenyl)amide;
N-[2(R)-Nonylsuccinic acid]-L-norvaline-N-(4-methoxyphenyl)amide;
N-[2(R)-Nonylsuccinic acid]-L-arginine-N-(4-methoxyphenyl)amide;
N-[2(R)-Nonylsuccinic acid]-L-phenylglycine-N-methylamide;
N-[2(R)-Nonylsuccinic acid]-L-tyrosine-N-cyclopentylamide; and
N-[2(R)-Nonylsuccinic acid]-L-tyrosine-N-3-dimethylaminopropylamide.

3. A compound according to claim 1 selected from the group consisting of:
N-[2(R)-Nonylsuccinic acid]-L-tyrosine-N-(2-morpholinesulfonylamino)ethylamide;
N-[2(R)-Nonylsuccinic acid]-L-phenylalanine-N-(2-morpholinesulfonylamino)ethylamide;
N-[2(R)-Nonylsuccinic acid]-L-valine-N-(2-morpholinesulfonylamino)ethylamide;
N-[2(R)-Nonylsuccinic acid]-L-tyrosine-N-(3-morpholinesulfonylamino)propylamide;
N-[2(R)-Nonylsuccinic acid]-L-phenylalanine-N-(3-morpholinesulfonylamino)propylamide;
N-[2(R)-Nonylsuccinic acid]-L-valine-N-(3-morpholinesulfonylamino)propylamide;
N-[2(R)-Nonylsuccinic acid]-L-phenylglycine-N-(3-morpholinesulfonylamino)propylamide;
N-[2(R)-Nonylsuccinic acid]-L-phenylglycine-N-(2-morpholinesulfonylamino)ethylamide;

4. A method of treating pain by administering an MMP-2/MMP-9 inhibitor according to claim 1.

5. A method according to claim 4 wherein the compound is selected from the group consisting of:
N-[2(R)-Nonylsuccinic acid]-L-tyrosine-N-3-(N-morpholino)propylamide;
N-[2(R)-Nonylsuccinic acid]-L-phenylglycine-N-3-(N-morpholino)propylamide;
N-[2(R)-Nonylsuccinic acid]-L-leucine-N-3-(N-morpholino)propylamide;
N-[2(R)-Nonylsuccinic acid]-L-methionine-N-3-(N-morpholino)propylamide;
N-[2(R)-Nonylsuccinic acid]-L-tyrosine-N-2-(N-morpholino)ethylamide;
N-[2(R)-Nonylsuccinic acid]-L-phenylalanine-N-3-(N-morpholino)propylamide;
N-[2(R)-Nonylsuccinic acid]-L-valine-N-2-(N-morpholino)ethylamide;
N-[2(R)-Nonylsuccinic acid]-L-tyrosine-N-(4-methoxyphenyl)amide;
N-[2(R)-Nonylsuccinic acid]-L-phenylalanine-N-(4-methoxyphenyl)amide;
N-[2(R)-Nonylsuccinic acid]-L-norvaline-N-(4-methoxyphenyl)amide;
N-[2(R)-Nonylsuccinic acid]-L-arginine-N-(4-methoxyphenyl)amide;
N-[2(R)-Nonylsuccinic acid]-L-phenylglycine-N-methylamide;
N-[2(R)-Nonylsuccinic acid]-L-tyrosine-N-cyclopentylamide; and
N-[2(R)-Nonylsuccinic acid]-L-tyrosine-N-3-dimethylaminopropylamide.

6. A method according to claim 5 wherein the disease treated is selected from the group consisting of stroke; hemorrhage; reperfusion injury; cerebral ischemia; cerebral infarction; enhanced or exaggerated sensitivity to pain, such as hyperalgesia, causalgia and allodynia; acute pain; burn pain; atypical facial pain; neuropathic pain; back pain; complex regional pain syndromes I and II; arthritic pain; sports injury pain; pain related to viral infection, e.g., HIV, post-polio syndrome, and post-herpetic neuralgia; phantom limb pain; labor pain; cancer pain; post-chemotherapy pain; post-stroke pain; post-operative pain; physiological pain; inflammatory pain; acute inflammatory conditions/visceral pain, e.g., angina, irritable bowel syndrome (IBS), and inflammatory bowel disease; neuropathic pain; neuralgia; painful diabetic neuropathy; traumatic nerve injury; spinal cord injury; and tolerance to narcotics or withdrawal from narcotics.
